(19) United States
(12) Patent Application Publication
(10) Pub. No.: US 2012/0329045 A1
(43) Pub. Date: Dec. 27, 2012

(54) RISK ASSESSMENT FOR PHENYTOIN-INDUCED ADVERSE DRUG REACTIONS

(75) Inventors: Wen-Hung Chung, Taoyuan (TW); Shuen-Iu Hung, Taoyuan (TW)

(73) Assignee: Chang Gung Medical Foundation Chang Gung Memorial Hospital at Keelung, Keelung (TW)

(21) Appl. No.: 13/323,515

(22) Filed: Dec. 12, 2011

Related U.S. Application Data
(63) Continuation-in-part of application No. PCT/CN2011/076187, filed on Jun. 23, 2011.

(51) Int. Cl. C12Q 1/68

(52) U.S. Cl. 435/6.11

(57) ABSTRACT

A method of predicting the risk of a patient for developing phenytoin-induced adverse drug reactions (ADRs), including Stevens-Johnson syndrome (SJS), toxic epidermal necrolysis (TEN), or drug reactions with eosinophilia and systemic symptoms (DRESS) is disclosed. Genetic polymorphisms of CYP2C genes (including CYP2C9, CYP2C19, CYP2C8 and CYP2C18), HLA alleles (including HLA-A*0207, HLA-A*2402, HLA-B*1301, HLA-B*1502, HLA-B*4001, HLA-B*4609, HLA-B*5101, HLA-DRB1*1001 or HLA-DRB1*1502) and phenytoin concentration in the patient's plasma can all contribute to phenytoin-induced ADRs.

### FIELD OF THE INVENTION

The present invention is related to a method for predicting the risk of a patient for adverse drug reactions, and more particularly to a risk assessment for developing adverse drug reactions in response to phenytoin.

### BACKGROUND OF THE INVENTION

Adverse drug reactions (abbreviated ADRs) are expressions that describe harm associated with the use of given medication at a normal dosage. Drug hypersensitivity is a common adverse event during medical treatments and contributes to about 20% of reported ADRs. These hypersensitivity reactions may present from mild skin rash (MPE) to life-threatening drug reaction with eosinophilia and systemic symptoms (DRESS), Stevens-Johnson syndrome (SJS), or toxic epidermal necrolysis (TEN).

Many aromatic antiepileptic drugs (AEDs), such as phenytoin, carbamazepine or lamotrigine, are frequently associated with hypersensitive reactions. In particular, phenytoin is a first-line AED, however, more than 19% of patients received phenytoin developed hypersensitivity reactions. Previous studies showed that the deficiency of microsomal epoxide hydroxylase and human leukocyte antigen (HLA) subtypes may associate with AED hypersensitivity. However, the relationship between drug metabolism/genetic susceptibility and phenytoin-induced hypersensitivity reactions is still unclear.

Therefore, there remains a need for a new and improved method for predicting the risk of phenytoin-induced hypersensitivity, wherein the risk can be assessed by evaluating the factors including genetic polymorphisms of CYP2C9, phenytoin plasma concentration and HLA genotypes.

### SUMMARY OF THE INVENTION

The present invention provides a method for accessing the risk of a patient for developing phenytoin-induced adverse drug reactions, particularly for SJS, TEN, and DRESS that are severe cutaneous adverse reactions. By using genome wide association study (Affymetrix 6.0) of 58 phenytoin-induced severe cutaneous adverse drug reactions (SCARs), including SJS, TEN, DRESS and 198 controls. In one embodiment, the method for accessing the risk of a patient for developing phenytoin-induced adverse drug reactions comprises the step of detecting the presence of SNPs (Single Nucleotide Polymorphism) on chromosome 10 in the CYP2Cs region associated with phenytoin-induced hypersensitivity, such as rs17110192, rs7896133 on CYP2C18; rs1057910 (CYP2C9*3), rs17110321, rs9332093, rs9332245 on CYP2C9; rs3758581, rs2860905, rs4086116 on CYP2C19 and rs7899038, rs1592037, rs1934952, rs11572139, rs6583967 on CYP2C8.

In another embodiment, the method for accessing the risk of a patient for developing phenytoin-induced adverse drug reactions comprises the step of combining rs1057910 (CYP2C9*3), rs3758581, rs17110192, rs9332245 or rs1592037 (especially for rs17110192, rs1057910 and rs3758581) belonging to a haplotype that can increase the statistical significance of association with phenytoin-induced SJS/TEN/DRESS comparing to phenytoin tolerant controls. There was no phenytoin tolerant individuals carried the combination of rs1057910, rs3758581, rs17110192, rs9332245 or rs1592037 (especially for rs1057910 and rs3758581, 0/95), however, near one third of phenytoin hypersensitivity patients carried this combination (30/96). Some of these SNPs cause a change in the amino acid sequence. For example, rs3758581, a missense change in CYP2C19 exon7 was significantly associated with phenytoin-SJS/TEN/DRESS. It is further discovered that CYP2C9*3 has a strong association with phenytoin-induced SJS/TEN/DRESS.

In another embodiment, the present invention provides a method for accessing the risk of a patient for developing phenytoin-induced adverse drug reactions comprising utilizing a candidate genes (CYP2Cs and HLA) approach of 152 patients with phenytoin-induced hypersensitivity reactions, including 53 cases of phenytoin-SJS/TEN, 24 cases of phenytoin-DRESS and 75 cases of phenytoin-MPE as well as 118 tolerant controls that received phenytoin more than 3 months without any adverse reaction. The result showed there are up to 30.2% of phenytoin-SJS/TEN and 37.5% of phenytoin-DRESS carried CYP2C9*3 genotype. In comparison, 14.7% of phenytoin-MPE and only 2.5% of tolerant controls carried CYP2C9*3. The present invention also indicates that genetic variants in CYP2C9 as well as in CYP2C19, CYP2C8 and CYP2C18 are related to poor metabolic activity for phenytoin and increase drug accumulation in plasma that leads to hypersensitivity reactions. In addition to the genetic link in CYP2Cs, HLA-A*0207, HLA-A*2402, HLA-B*1301, HLA-B*1502, HLA-B*4001, HLA-B*4609, HLA-B*5101, HLA-DRB1*1001 or HLA-DRB1*1502 also showed significant association with phenytoin-induced hypersensitivity. Thus, these data revealed that the genetic polymorphisms of CYP2C9/2C19/2C8/2C18 or its defective alleles, the phenytoin plasma levels and specific HLA genotypes all contribute to phenytoin induced severe cutaneous adverse drug reactions.

### BRIEF DESCRIPTION OF THE DRAWINGS

FIG. 1 illustrates the genome-wide association study of phenytoin-induced SCARs, where each dot represents a SNP. The x-axis represents the position of the SNP on chromosomes, while the y-axis represents the −log₁₀ P value of the SNP in the case-control association study. 58 phenytoin-induced SJS/TEN/DRESS and 198 population controls are included in the study. SNPs with P values <10⁻⁶ is highlighted in red. The strong signal in chromosome 10 lies in the CYP2C region.

FIG. 2 shows phenytoin plasma level of phenytoin-ADRs and tolerant controls, where the normal therapeutic levels of phenytoin are usually between 10-20 μg/ml and the phenytoin plasma levels of tolerant controls were between 5-15 μg/ml. However, the phenytoin plasma levels of patients with phenytoin-SJS/TEN were 40-50 μg/ml that were higher than that of tolerant controls.

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

All publications mentioned are incorporated by reference for the purpose of describing and disclosing, for example, the designs and methodologies that are described in the publications that might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

The diagnosis of Stevens-Johnson syndrome (SJS) and toxic epidermal necrolysis (TEN) were based on the clinical morphology defined by consensus criteria (Bastuji-Garin S, Rzany B et al, 1993). SJS is defined as skin detachment of <10% of body-surface area, overlap SJS_TEN as skin detachment of 10-29% and TEN as >/=30%. The criteria for DRESS were cutaneous rash (e.g. diffuse macuopapular, exfoliative dermatitis) with symptoms of eosinophilia, atypical circulating lymphocytes, acute hepatocellular injury, or worsening renal function (Kardaun S H, Sidoroff A et al, 2007). Patients who fulfilled the diagnostic criteria of SJS, TEN, or DRESS induced by phenytoin were identified in Chang Gung Memorial Hospital Health System and were enrolled for this study.

It was discovered that specific CYP2C genetic variants, including CYP2C9, CYP2C19, CYP2C8 and CYP2C18 were related to low metabolic activity of phenytoin and was associated with phenytoin-induced hypersensitivity reactions. In addition to CYP2C variants, HLA-A*0207, HLA-A*2402, HLA-B*1301, HLA-B*1502, HLA-B*4001, HLA-B*4609, HLA-B*5101, HLA-DRB1*1001 or HLA-DRB1*1502 were also discovered to show significant association with phenytoin-induced hypersensitivity or cutaneous adverse drug reactions. It is noted that “phenytoin” in the present invention may also include phenytoin-like anticovulsant drugs including fosphenytoin, phenobarbital, lamotrigine, carbamazepine and oxcarbazepine.

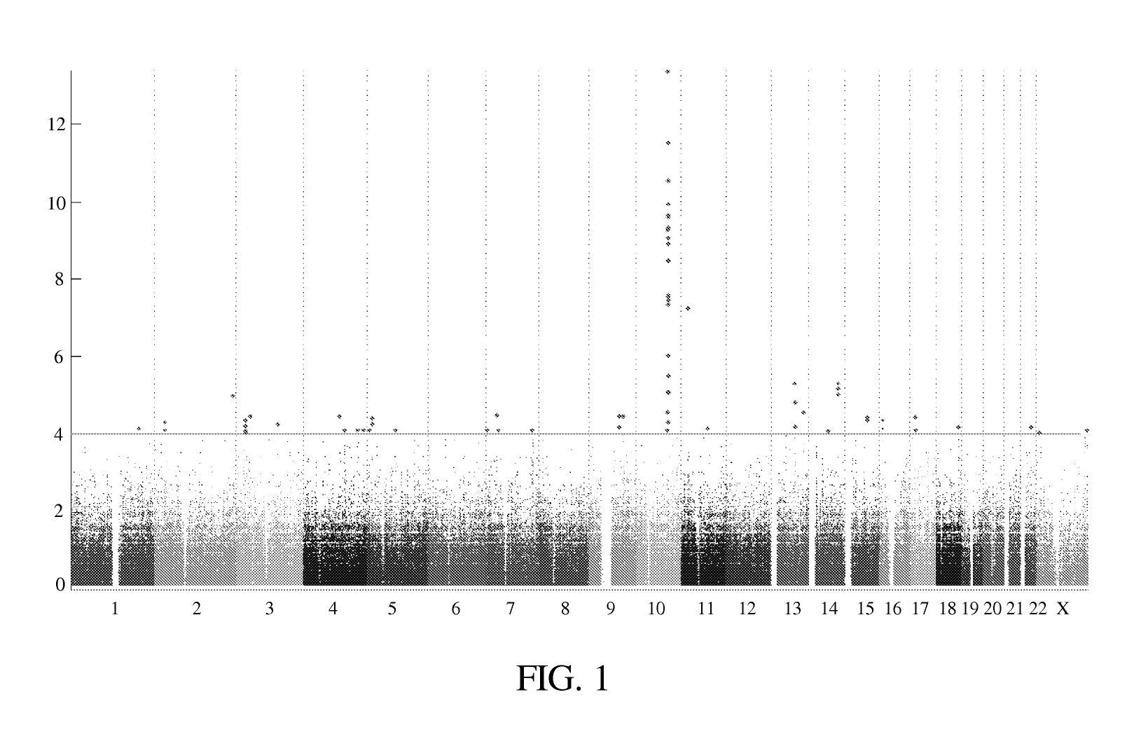

Thus, the present invention provides a method of assessing the risk of phenytoin-induced adverse drug reactions comprising the step of determining the presence of SNPs (Single Nucleotide Polymorphism) in chromosome 10 of the CYP2C region, including CYP2C9, CYP2C19, CYP2C8 and CYP2C18. By using genome wide association study (Affymetrix 6.0) of 58 phenytoin-induced SCARs (including SJS, TEN, and DRESS) and 198 population controls, it is found that the most significant SNPs were in chromosome 10 (see FIG. 1) of the CYP2C region, such as rs17110192, rs7896133 on CYP2C18; rs17110321, rs9332093, rs9332245 on CYP2C9; rs3758581, rs2860905, rs4086116 on CYP2C19 and rs7899038, rs1592037, rs1934952, rs11572139, rs6583967 on CYP2C8 (see Table 1). The other SNPs which located nearby CYP2C loci, such as rs2274222, rs11188183, rs7921561, rs10882544, rs7084271, rs644437, rs12769577, rs617848, rs10882551, rs585381, rs648638, rs664093, rs12262878, rs17524438, rs12413028, rs11188246, rs12415795, rs11596107, rs11596737, rs10509685, rs7912686, rs17453729, rs17453764, rs17526000 and rs12769370 were also significantly associated with phenytoin-induced hypersensitivity reactions (see Table 1).

Previous study indicated that CYP2C9*3 increased the risk of phenytoin-induced maculopapular exanthem (MPE) (Lee A Y. et al. 2004). In one embodiment, CYP2C9*3 is used to predict the risk of phenytoin-induced adverse drug reactions. To examine the role of CYP2C9*3 on other types of phenytoin-induced adverse drug reactions (ADRs), especially for the unique mucocutaneous blistering phenotypes of SJS or TEN, a single nucleotide polymorphism (SNP) geno typing has been conducted on patients with adverse drug reactions induced by phenytoin. 152 patients were enrolled for the study of phenytoin-induced hypersensitivity reactions, including 53 cases of phenytoin-SJS/TEN, 24 cases of phenytoin-DRESS and 75 cases of phenytoin-MPE, as well as 118 tolerant controls that received phenytoin more than 3 months without any adverse reaction. The results showed that CYP2C9*3 variant was present in 16 of 53 (30.2%) phenytoin-induced SJS/TEN patients, 9 of 24 (37.5%) phenytoin-induced DRESS patients and 11 of 75 (14.7%) phenytoin-induced MPE. In comparison, the variant was only found in 2.5% (3/118) of the phenytoin-tolerant group (see Table 2). Statistical analysis of phenytoin hypersensitivity cases and tolerant controls showed that CYP2C9*3 was most significantly associated with phenytoin induced SJS/TEN (SJS/TEN vs. tolerant controls P=3.3×10⁻⁷, OR=17.5 (4.8-63.7) or DRESS (DRESS vs. tolerant controls P=6.1×10⁻⁵, OR=19.2 (4.4-82.7)), but only weakly associated with phenytoin-MPE (MPE vs. tolerant control P=0.004, OR=6.2 (1.6-23.3)), suggesting that the presence of this CYP2C9*3 variant can be used in the identification of high-risk patients for phenytoin-induced ADRs, particularly phenytoin-induced SJS/TEN or DRESS.

In another embodiment, the SNPs on CYP2C causing the amino acid to change can also be used for assessing the risk of phenytoin-induced adverse drug reactions. A further study has been conducted for some SNPs on CYP2C genes which can make amino acid changed, such as 371G>A (rs12414460, Arg124Gln), 895A>G, (rs72558192, Thr299Ala, known as CYP2C9*16), 1362G>C (Gln454H is, known as CYP2C9*19) on CYP2C9; 991A>G (rs3758581, Ile331Val), 395G>A (rs72558184, Arg132Gln, known as CYP2C19*6), 636G>A (rs4986893, Trp212end, known as CYP2C19*3), 681G>A, (rs4244285, causing splicing site mutation, known as CYP2C19*2), 781C>T (Arg261Trp) on CYP2C19 and 204T>A (rs41291550 Tyr68end), 370C>T (Arg124Trp), 576G>C (Gln192H is), 1154C>T (rs2281891, Thr385Met) on CYP2C18. These changes of CYP2C enzyme can affect both its activity and its substrate specificity. For example, rs3758581, a missense change in CYP2C19 exon7 was significantly associated with phenytoin-SJS/TEN/DRESS (SJS/TEN/DRESS vs. Tolerant: P=0.0003, OR=7.28 (2.3564-22.4912)) (see Table 1).

TABLE 1
List of SNPs of CYP2C loci in chromosome 10 significantly
associated with phenytoin-SJS/TEN/DRESS

| | | | % A, B | |
|---|---|---|---|---|
| RS# | Annotation | Chr | Fisher_P | OR |
| rs17110192 | CYP2C18 | 10 | 4.03E−14 | 13.33333 |
| rs7896133 | CYP2C18 | 10 | 1.14E−06 | 0.115132 |
| rs17110321 | CYP2C9 | 10 | 4.03E−14 | 0.075 |
| rs9332093 | CYP2C9 | 10 | 1.19E−13 | 13.00438 |
| rs9332245 | CYP2C9 | 10 | 4.37E−13 | 12.5 |
| rs2860905 | CYP2C19 | 10 | 1.14E−06 | 0.115132 |
| rs4086116 | CYP2C19 | 10 | 3.49E−05 | 7.6 |
| rs3758581 | CYP2C19 | 10 | 3.00E−04 | 7.28 |
| rs7899038 | CYP2C8 | 10 | 1.87E−07 | 0.078083 |
| rs1592037 | CYP2C8 | 10 | 4.92E−13 | 11.67059 |
| rs1934952 | CYP2C8 | 10 | 7.39E−10 | 6.441558 |
| rs11572139 | CYP2C8 | 10 | 6.38E−06 | 15.32967 |
| rs6583967 | CYP2C8 | 10 | 4.92E−13 | 11.67059 |
| rs11188183 | chromosome 10 open | 10 | 3.06E−12 | 0.083365 |
| | reading frame 129 | | | |
| rs7921561 | chromosome 10 open | 10 | 5.52E−13 | 11.6098 |
| | reading frame 129 | | | |
| rs644437 | chromosome 10 open | 10 | 4.92E−13 | 11.67059 |
| | reading frame 129 | | | |
| rs12769577 | chromosome 10 open | 10 | 4.92E−13 | 11.67059 |
| | reading frame 129 | | | |
| rs10882551 | chromosome 10 open | 10 | 4.96E−14 | 14.61539 |
| | reading frame 129 | | | |
| rs585381 | chromosome 10 open | 10 | 4.31E−12 | 0.096042 |
| | reading frame 129 | | | |
| rs648638 | chromosome 10 open | 10 | 4.92E−13 | 11.67059 |
| | reading frame 129 | | | |
| rs664093 | chromosome 10 open | 10 | 4.92E−13 | 0.085685 |
| | reading frame 129 | | | |
| rs12262878 | chromosome 10 open | 10 | 1.67E−13 | 12.76471 |
| | reading frame 129 | | | |
| rs17524438 | chromosome 10 open | 10 | 1.26E−12 | 0.0875 |
| | reading frame 129 | | | |

TABLE 2
CYP2C9*3 frequency in 152 patients with phenytoin-induced
cutaneous ADRs and 118 tolerant controls.

| CYP2C9 Genotypes | CYP2C9*1 | CYP2C9*3^{§} | Total |
|---|---|---|---|
| SJS/TEN | 37 (69.8%) | 16 (30.2%) | 53 |
| DRESS | 15 (62.5%) | 9 (37.5%) | 24 |
| MPE | 64 (85.3%) | 11 (14.7%) | 75 |
| Tolerance | 115 (97.5%) | 3 (2.5%) | 118 |

^{§}SJS/TEN vs. Tolerant: P = 3.3 × 10⁻⁷, OR = 17.5 (4.8-63.7); DRESS vs. Tolerant: P = 6.1 × 10⁻⁵, OR = 19.2 (4.4-82.7); MPE vs. Tolerant: P = 0.004, OR = 6.2 (1.6-23.3)

In still another embodiment in the present invention, different SNPs can be combined for assessing the risk of phenytoin-induced adverse drug reactions. It is found that a combination of rs1057910 (CYP2C9*3), rs3758581, rs17110192, rs9332245 or rs1592037 belonging to a haplotype can increase the statistical significance of association with phenytoin-induced SJS/TEN/DRESS comparing to phenytoin tolerant controls. Especially when patients carry both rs1057910 and rs3758581 or rs17110192 have more significant association with phenytoin-induced SJS/TEN/DRESS (see Table 3).

TABLE 3
Statistical analysis of major SNPs associated with phenytoin-induced
SJS/TEN/DRESS

| | | PHT-Case | | Control | | | | | |
|---|---|---|---|---|---|---|---|---|---|
| | | (n = 26) | | (n = 95) | | Fisher's test | Chi-square | Odds ratio | 95% CI |
| 6 SNPs | rs1592037, rs9332245, | 2 | 124 | 0 | 95 | 0.50761004 | 0.21731708 | 3.0645 | 0.137-68.746 |
| | rs1057910, rs3758581, | | | | | | | | |
| | rs4986893, rs17110192 | | | | | | | | |
| 5 SNPs | rs1592037, rs9332245, | 29 | 97 | 0 | 95 | 1.93E−08 | 5.30E−07 | 56.8041 | 3.4201-943.446 |
| | rs1057910, rs3758581, | | | | | | | | |
| | rs17110192 | | | | | | | | |
| 4 SNPs | rs1592037, rs1057910, | 29 | 97 | 0 | 95 | 1.93E−08 | 5.30E−07 | 56.8041 | 3.420-943.446 |
| | rs3758581, rs17110192 | | | | | | | | |
| | rs1592037, rs9332245 | 31 | 95 | 1 | 94 | 9.93E−08 | 8.40E−07 | 30.6737 | 4.103-229.311 |
| | rs1057910, rs17110192 | | | | | | | | |
| | rs1592037, rs9332245, | 29 | 97 | 2 | 93 | 2.7454E−06 | 0.00000936 | 13.9021 | 3.226-59.915 |
| | rs3758581, rs17110192 | | | | | | | | |
| 3 SNPs | rs1057910, rs3758581, | 30 | 96 | 0 | 95 | 9.50E−09 | 3.10E−07 | 59.375 | 3.578-985.443 |
| | (rs17110192/rs9332245) | | | | | | | | |
| | rs9332245, rs17110192, | 29 | 97 | 3 | 92 | 1.3629E−05 | 0.0000328 | 9.1684 | 2.700-31.131 |
| | (rs3758581/rs1592037) | | | | | | | | |
| | rs1592037, rs9332245, | 29 | 97 | 2 | 93 | 2.7454E−06 | 0.00000936 | 13.9021 | 3.226-59.915 |
| | rs3758581 | | | | | | | | |
| | rs1592037/rs9332245/ | 30 | 96 | 3 | 92 | 7.3945E−06 | 0.00002005 | 9.5833 | 2.827-32.487 |
| | rs3758581/rs17110192 | | | | | | | | |
| 2 SNPs | rs1057910, rs17110192 | 31 | 95 | 1 | 94 | 9.93E−08 | 8.40E−07 | 30.6737 | 4.103-229.311 |
| | rs1057910, rs3758581 | 30 | 96 | 0 | 95 | 9.50E−09 | 3.10E−07 | 59.375 | 3.5778-985.443 |
| | rs1057910, (rs1592037/ | 30 | 96 | 1 | 94 | 2.05E−07 | 1.42E−06 | 29.375 | 3.926-219.805 |
| | rs9332245/rs758581) | | | | | | | | |

In a further embodiment, the present invention provides a method of assessing the risk of phenytoin-induced adverse drug reactions comprising the step of identifying the presence of HLA genotypes, including HLA-A, HLA-B and HLA-DRB1. PCR-SSO is used to identify the HLA-A, HLA-B and HLA-DRB1 genotype of phenytoin-ADRs patients and tolerant controls. The results indicated that HLA-A*0207, HLA-A*2402, HLA-B*1301, HLA-B*1502, HLA-B*4001, HLA-B*4609, HLA-B*5101, HLA-DRB1*1001 or HLA-DRB1*1502 were significantly associated with phenytoin-induced ADRs (SJS/TEN, DRESS, or MPE) (see Table 4 to 6). The data shows that HLA-B*1301 allele significantly increased frequencies among patients with phenytoin-ADRs compared to the tolerant controls. There were 15 SJS/TEN patients (28.3%) and 12 DRESS patients (46.2%) carried HLA-B*1301, while only 14 tolerant patients (11.9%) carried this genotype (SJS/TEN vs. tolerant controls: P=0.001, OR=3.8 (1.7-8.5); DRESS vs. tolerant controls: P=2×10⁻⁴, OR=6.4 (2.5-16.5)). However, this association was not significant in MPE (MPE vs. Tolerant controls: P=0.296, OR=1.7 (0.7-3.7)). HLA-B*5101 allele was also associated with phenytoin-induced ADRs. There were 13.2% SJS/TEN patients, 19.2% DRESS patients and 15.6% MPE patients carried HLA-B*5101, while only 5 tolerant controls (4.2%) carried this genotype (SJS/TEN vs. tolerant controls: P=0.05, OR=3.4; DRESS vs. tolerant controls: P=0.018, OR=5.4; MPE vs. tolerant controls: P=0.009, OR=4.2).

TABLE 4
Associations between HLA-A alleles and phenytoin-induced cutaneous ADR

| | | | | Odds | | | Odds | | | Odds |
|---|---|---|---|---|---|---|---|---|---|---|
| HLA-A | Tolerant | SJS/TEN | p- | ratio | DRESS | p- | ratio | MPE | | ratio |
| allele | (n = 117) | (n = 53) | value | (95% CI) | (n = 22) | value | (95% CI) | (n = 75) | p-value | (95% CI) |
| 0207 | 7 | 7 | 0.135 | 2.4 | 5 | 0.024 | 4.6 | 9 | 0.182 | 2.1 |
| | (6.0%) | (13.2%) | | (0.8-7.2) | (22.7%) | | (1.3-16.2) | (12.0%) | | (0.8-6.0) |
| 2402 | 38 | 8 | 0.025 | 0.4 | 4 | 0.214 | 0.5 | 20 | 0.424 | 0.8 |
| | (32.5) | (15.1%) | | (0.2-0.9) | (18.2%) | | (0.1-1.5) | (26.7%) | | (0.4-1.4) |

TABLE 5
Associations between HLA-B alleles and phenytoin-induced cutaneous ADR

| | | | | Odds | | | Odds | | | Odds |
|---|---|---|---|---|---|---|---|---|---|---|
| HLA-B | Tolerant | SJS/TEN | p- | ratio | DRESS | p- | ratio | MPE | | ratio |
| allele | (n = 118) | (n = 53) | value | (95% CI) | (n = 26) | value | (95% CI) | (n = 77) | p-value | (95% CI) |
| 1301 | 14 | 15 | 0.001 | 3.8 | 12 | 2 × 10⁻⁴ | 6.4 (2.5-16.5) | 14 | 0.296 | 1.7 (0.7-3.7) |
| | (11.9%) | (28.3%) | | (1.7-8.5) | (46.2%) | | | (18.2%) | | |
| 1502 | 9 | 12 | 0.010 | 3.5 | 0 | 0.213 | 0.2 (0.0-4.2) | 8 | 0.605 | 1.4 (0.5-3.8) |
| | (7.6%) | (22.6%) | | (1.4-9.0) | (0.0%) | | | (10.4%) | | |
| 5101 | 5 | 7 | 0.050 | 3.4 | 5 | 0.018 | 5.4 (1.4-20.2) | 12 | 0.009 | 4.2 (1.4-12.4) |
| | (4.2%) | (13.2%) | | (1.0-11.4) | (19.2%) | | | (15.6%) | | |
| 4609 | 0 | 0 | 1.000 | — | 2 | 0.032 | 19.7 (0.9-449.8) | 0 | 1.000 | — |
| | (0%) | (0%) | | | (7.7%) | | | (0%) | | |
| 4001 | 52 | 20 | 0.504 | 0.8 | 9 | 0.393 | 0.7 (0.3-1.6) | 22 | 0.035 | 0.5 (0.3-1.9) |
| | (44.1%) | (37.7%) | | (0.4-1.5) | (34.6%) | | | (28.6%) | | |

Our previous study has showed that HLA-B*1502 allele was strongly associated with carbamazepine-induced SJS/TEN (Chung W H et al, 2004). In still a further embodiment of the present invention, HLA-B*1502 allele is used for assessing the risk of phenytoin-induced adverse drug reactions, and the results showed that HLA-B*1502 was associated with phenytoin-induced ADRs. There were 12 SJS/TEN patients (22.6%) carried HLA-B*1502, while only 9 tolerant controls (7.6%) carried this genotype. HLA-B*1502 allele significantly increased frequencies among the phenytoin-induced SJS/TEN patients compared to the tolerant controls (SJS/TEN vs. tolerant: P=0.01, OR=3.5 (1.4-9.0)). However, this association was not seen in phenytoin-induced DRESS and MPE (P>0.05).

In addition, in the present invention, we also found that HLA-B*4609 allele significantly increased frequencies among the phenytoin-induced DRESS patients compared to the tolerant controls (DRESS vs. tolerant controls: P=0.032, OR=19.7 (0.9-449.8)) and HLA-A*0207 allele was also associated with phenytoin-induced DRESS (DRESS vs. tolerant controls: P=0.024, OR=4.6 (1.3-16.2)). Moreover, HLA-DRB1*1001 was found to be associated with phenytoin-induced MPE (MPE vs. tolerant controls: P=0.02, OR=13.2); HLA-DRB1*1502 was found to be associated with phenytoin-induced SJS/TEN (SJS/TEN vs. tolerant controls: P=0.029, OR=14). In contrast, HLA-A*2402 and HLA-B*4001 allele were showed a protective effect in phenytoin-induced ADRs (decreased allele frequencies in phenytoin-induced ADRs, increased in tolerant controls).

TABLE 6
Associations between HLA-DRB1 alleles and phenytoin-induced cutaneous ADRs

| HLA- | | | | Odds | | | Odds | | | Odds |
|---|---|---|---|---|---|---|---|---|---|---|
| DRB1 | Tolerant | SJS/TEN | p- | ratio | DRESS | p- | ratio | MPE | p- | ratio |
| allele | (n = 117) | (n = 53) | value | (95% CI) | (n = 22) | value | (95% CI) | (n = 75) | value | (95% CI) |
| 1001 | 0 (0.0%) | 0 | 1.000 | — | 0 | 1.000 | — | 4 | 0.022 | 13.2 |
| | | (0.0%) | | | (0.0%) | | | (5.3%) | | (0.7-253.1) |
| 1502 | 0 (0.0%) | 3 | 0.029 | 14.0 | 0 | 1.000 | — | 0 | 1.000 | — |
| | | (5.7%) | | (0.7-285.5) | (0.0%) | | | (0.0%) | | |

The study also indicates that genetic variants in CYP2C9 as well as in CYP2C19, CYP2C8, CYP2C18 are related to poor metabolic activity for phenytoin and increase its drug accumulation that leads to the risk of phenytoin hypersensitivity. In some embodiments, the method for assessing the risk of phenytoin-induced adverse drug reactions comprises the step of detecting phenytoin concentration in plasma. Since phenytoin is metabolized by liver enzymes, polymorphisms of cytochrome P450 enzymes can affect phenytoin plasma concentrations. The non-linear pharmacokinetics of phenytoin and narrow therapeutic window suggesting phenytoin concentrations in plasma may be related to its efficacy and toxicity.

In this study, the phenytoin concentrations during ADR onset were determined from patients' plasma by HPLC analysis and estimated by non-linear pharmacokinetics formula:

$$t = \frac{{K}_{m} \times ln ( \frac{{C}_{1}}{{C}_{2}} ) + ( {C}_{1} - {C}_{2} )}{{V}_{max}} \times {V}_{d}$$

Km=4 mg/L (substrate concentration at which the rate of metabolism is one half of Vmax)
Vmax=7 mg/kg/day (maximum metabolic rate)
Vd=0.65 L/kg (volume of distribution)
t: the time (days) between any two phenytoin plasma concentration

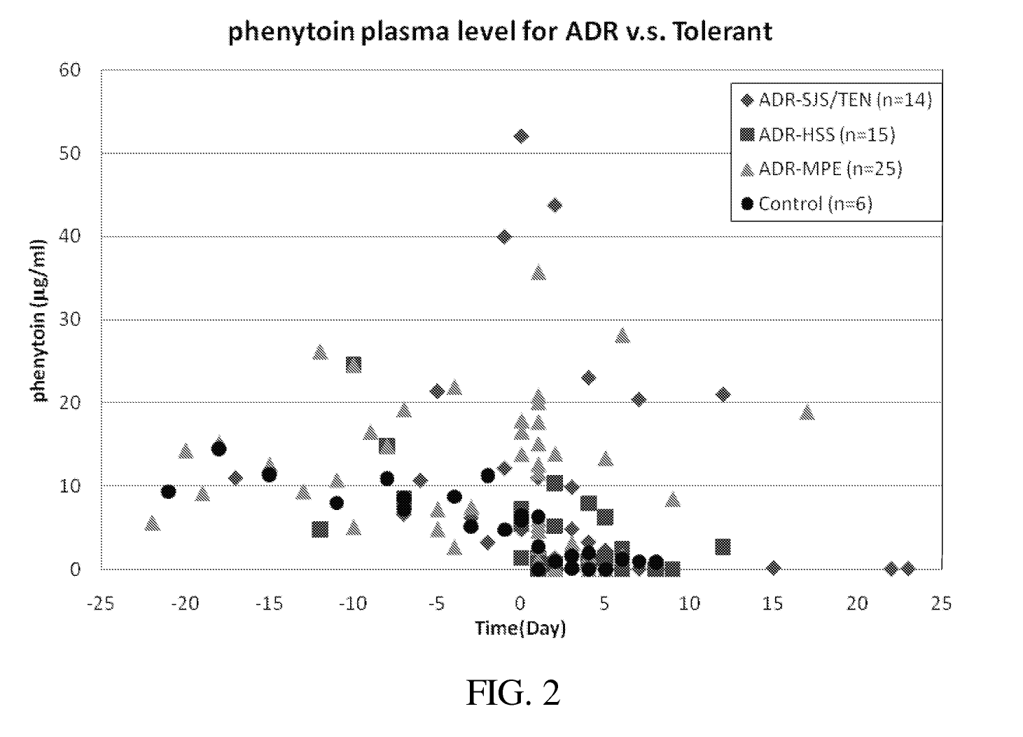

Therapeutic levels of phenytoin are usually between 10-20 μg/ml. After taking drug, phenytoin plasma levels of tolerant control patients were between 5-15 μg/ml. However, in SJS/TEN patients, the phenytoin plasma levels were up to 40-50 μg/ml that were higher than tolerant control patients (SJS/TEN vs. tolerant control: P=0.006; MPE vs. tolerant controls: P=0.004) (see FIG. 2). We also compared the phenytoin plasma levels of patients who carried CYP2Cs variants. The result showed that phenytoin-ADRs patients who carried CYP2C9*3 were with higher phenytoin plasma levels. After analyzing the phenytoin level of 23 phenytoin-ADRs patients, there were 4 patients whose phenytoin levels were lower than 10 μg/ml, 9 patients' phenytoin levels were between 10-20 μg/ml and 10 patients' phenytoin levels were higher than 20 μg/ml. Among these 23 patients, the phenytoin concentrations of 5 patients who carry CYP2C9*3 variant were all higher than 20 μg/ml. The invention proves CYP2Cs variants, especially CYP2C9*3, display poor metabolic activity of phenytoin and can result in hypersensitivity reactions. Defective metabolism of phenytoin may increase the risk of drug accumulation, and leads to its risk to induce severe hypersensitivity reactions, such as SJS/TEN and DRESS.

The CYP2Cs variants and HLA genotypes can be detected by using any method known in the art. Preferably, genomic DNA is hybridized to a probe that is specific for the variant of interest. The probe may be labeled for direct detection, or contacted by a second, detectable molecule that specifically binds to the probe. Alternatively, cDNA, RNA, or protein product of the variant can be detected.

Having described the invention by the description and illustrations above, it should be understood that these are exemplary of the invention and are not to be considered as limiting. Accordingly, the invention is not to be considered as limited by the foregoing description, but includes any equivalents.

### REFERENCES

1. Ingelman-Sundberg, M. Pharmacogenomic biomarkers for prediction of severe adverse drug reactions. N Engl J Med 358, 637-639 (2008).
2. Bohan, K. H., et al. Anticonvulsant hypersensitivity syndrome: implications for pharmaceutical care. Pharmacotherapy. 27(10), 1425-1439 (2007).
3. Odani, A., et al. Genetic polymorphism of the CYP2C subfamily and its effect on the pharmacokinetics of phenytoin in Japanese patients with epilepsy. Clin Pharmacol Ther 62, 287-292 (1997).
4. Shintani, et al. Genetic polymorphisms and functional characterization of the 5′-flanking region of the human CYP2C9 gene: In vitro and in vivo studies. Clin Pharmacol Ther. 70(2), 175-182 (2001).
5. Lee, A. Y., Kim, M. J., Chey, W. Y., Choi, J. & Kim, B. G. Genetic polymorphism of cytochrome P450 2C9 in diphenylhydantoin-induced cutaneous adverse drug reactions. Eur J Clin Pharmacol 60, 155-159 (2004).
6. Hung, S. I., et al. Common risk allele in aromatic antiepileptic-drug induced
Stevens-Johnson syndrome and toxic epidermal necrolysis in Han Chinese. Pharmacogenomics 11, 349-356 (2010).
7. Bastuji-Garin S, Rzany B, Stern R S, et al, Shear N H, Naldi L, Roujeau J C. Clinical classification of cases of toxic epidermal necrolysis, Stevens-Johnson syndrome, and erythema multiforme. Arch Dermatol 129, 92-6 (1993).
8. Kardaun S H, Sidoroff A, Valeyrie-Allanore L, Halevy S, Davidovici B B, Mockenhaupt M, et al. Variability in the clinical pattern of cutaneous side-effects of drugs with systemic symptoms: does a DRESS syndrome really exist? Br J Dermatol 156, 609-11 (2007).

What is claimed is:

1. A method of assessing the risk of a patient for developing phenytoin-induced adverse drug reactions (ADRs), comprising steps of:
detecting the presence of SNPs (Single Nucleotide Polymorphism) in or nearby CYP2C genes on chromosome 10 from a sample of the patient; and
associating the presence of SNPs in or nearby CYP2C genes on chromosome 10 with an increased risk for phenytoin-induced adverse drug reactions, which includes Stevens-Johnson syndrome (SJS), toxic epidermal necrolysis (TEN), drug reaction with eosinophilia and systemic symptoms (DRESS), or maculo-papular eruptions (MPE).

2. The method of assessing the risk of a patient for developing phenytoin-induced ADRs of claim 1, wherein the detecting the presence of SNPs in or nearby CYP2C genes includes a step of detecting SNPs on CYP2C variants including CYP2C9, CYP2C8, CYP2C18 and CYP2C19.

3. The method of assessing the risk of a patient for developing phenytoin-induced ADRs of claim 2, wherein the step of associating the presence SNPs in or nearby CYP2C on chromosome 10 with an increased risk for phenytoin-induced ADRs comprising a step of combining one or more SNPs to predict the risk for phenytoin-induced ADRs.

4. The method of assessing the risk of a patient for developing phenytoin-induced adverse drug reactions (ADRs) of claim 1, wherein the step of detecting the presence of genetic variants in or nearby CYP2C genes on chromosome 10 includes a step of using an oligonucleotide that specifically hybridizes with the nucleic acid coding for the variant.

5. The method of assessing the risk of a patient for developing phenytoin-induced adverse drug reactions (ADRs) of claim 1, wherein the step of detecting the presence of genetic variants in or nearby CYP2C genes on chromosome 10 includes a step of using DNA prepared from the peripheral blood of the patient.

6. A method of assessing the risk of a patient for developing phenytoin-induced adverse drug reactions (ADRs), comprising steps of:
detecting the presence of HLA alleles, including HLA-A, HLA-B and HLA-DRB1; and
associating the presence of the HLA alleles with an increased risk for phenytoin-induced adverse drug reactions, which includes Stevens-Johnson syndrome (SJS), toxic epidermal necrolysis (TEN), drug reaction with eosinophilia and systemic symptoms (DRESS), or maculo-papular eruptions (MPE).

7. The method of assessing the risk of a patient for developing phenytoin-induced adverse drug reactions (ADRs) of claim 6, wherein the step of detecting the presence of HLA alleles including a step of using PCR-SSO to identify HLA-A, HLA-B and HLA-DRB1 genotypes.

8. The method of assessing the risk of a patient for developing phenytoin-induced adverse drug reactions (ADRs) of claim 6, wherein the step of detecting the presence of HLA alleles includes a step of using an oligonucleotide that specifically hybridizes to the allele.

9. The method of assessing the risk of a patient for developing phenytoin-induced adverse drug reactions (ADRs) of claim 6, wherein the step of detecting the presence of HLA alleles includes a step of using DNA prepared from the peripheral blood of the patient.

10. A method of assessing the risk of a patient for developing phenytoin-induced adverse drug reactions (ADRs), comprising steps of:
detecting phenytoin concentration in the patient's plasma; and
associating the phenytoin concentration with an increased risk for phenytoin-induced adverse drug reactions, which includes Stevens-Johnson syndrome (SJS), toxic epidermal necrolysis (TEN), drug reaction with eosinophilia and systemic symptoms (DRESS), or maculo-papular eruptions (MPE).

11. The method of assessing the risk of a patient for developing phenytoin-induced adverse drug reactions (ADRs) of claim 10, wherein the step of detecting phenytoin concentration in the patient's plasma includes steps of using HPLC to analyze the phenytoin concentration and estimating the phenytoin concentration by non-linear pharmacokinetics formula:
$$t = \frac{{K}_{m} \times ln ( \frac{{C}_{1}}{{C}_{2}} ) + ( {C}_{1} - {C}_{2} )}{{V}_{max}} \times {V}_{d}$$

12. The method of assessing the risk of a patient for developing phenytoin-induced ADRs of claim 1, further comprising a step of detecting the change amino acids.

13. The method of assessing the risk of a patient for developing phenytoin-induced ADRs of claim 2, wherein the step of detecting SNPs on CYP2C variants (including CYP2C9, CYP2C8, CYP2C18 and CYP2C19) comprises a step of detecting the presence of CYP2C9*3 variants.

14. The method for assessing the risk of a patient for developing phenytoin-induced ADRs of claim 1, further comprising a step of administrating phenytoin-like anticovulsant drugs including fosphenytoin, phenobarbital, lamotrigine, carbamazepine and oxcarbazepine.

15. The method of assessing the risk of a patient for developing phenytoin-induced ADRs of claim 6, further comprising a step of administrating phenytoin-like anticovulsant drugs including fosphenytoin, phenobarbital, lamotrigine, carbamazepine and oxcarbazepine.

16. The method of assessing the risk of a patient for developing phenytoin-induced ADRs of claim 10, further comprising a step of administrating phenytoin-like anticovulsant drugs including fosphenytoin, phenobarbital, lamotrigine, carbamazepine and oxcarbazepine.

17. The method of assessing the risk of a patient for developing phenytoin-induced ADRs of claim 6, wherein the step of detecting the presence of HLA alleles (including HLA-A, HLA-B and HLA-DRB1) comprises a step of detecting the presence of HLA-A* 0207, 2402; HLA-B* 1301, 1502, 5101, 4609, 4001; and HLA-DRB1 1001, 1502.

18. The method of assessing the risk of a patient for developing phenytoin-induced ADRs of claim 7, wherein the step of using PCR-SSO to identify HLA-A, HLA-B and HLA-DRB1 genotypes comprises a step of identifying HLA-A* 0207, 2402; HLA-B* 1301, 1502, 5101, 4609, 4001; and HLA-DRB1 1001, 1502.
